(19) United States
(12) Patent Application Publication
(10) Pub. No.: US 2011/0196182 A1
(43) Pub. Date: Aug. 11, 2011

(54) STYRENE PRODUCTION PROCESSES AND CATALYSTS FOR USE THEREIN

(75) Inventor: JOSEPH THORMAN, Houston, TX (US)

(73) Assignee: Fina Technology, Inc., Houston, TX (US)

(21) Appl. No.: 13/010,134

(22) Filed: Jan. 20, 2011

Related U.S. Application Data
(60) Provisional application No. 61/301,765, filed on Feb. 5, 2010.

(51) Int. Cl. C07C 2/12; C07C 2/08

(52) U.S. Cl. 585/438; 585/435

(57) ABSTRACT

Styrene production processes and catalysts for use therein are described herein. The process generally includes providing a C₁ source; contacting the C₁ source with toluene in the presence of a catalyst disposed within a reactor to form a product stream including ethylbenzene, wherein the catalyst includes a nanocrystalline zeolite; and recovering the product stream from the reactor.

### FIELD

Embodiments of the present invention generally relate to methods for the production of styrene and ethylbenzene. More specifically, the embodiments relate to catalysts for use in such processes.

### BACKGROUND

Styrene is an important monomer used in the manufacture of many polymers. Styrene is commonly produced by forming ethylbenzene, which is then dehydrogenated to produce styrene. Ethylbenzene is typically formed by one or more aromatic conversion processes involving the alkylation of benzene.

Aromatic conversion processes, which are generally carried out utilizing a molecular sieve type catalyst, are well known in the chemical processing industry. Such aromatic conversion processes include the alkylation of aromatic compounds such as benzene with ethylene to produce alkyl aromatics, such as ethylbenzene. Unfortunately, such processes have been characterized by very low yields of desired products in addition to having very low selectivity to styrene and ethylbenzene.

In view of the above, it would be desirable to develop processes of forming styrene and/or ethylbenzene capable of increased yields and improved selectivity.

### SUMMARY

Embodiments of the present invention include styrene production processes. The process generally includes providing a C₁ source; contacting the C₁ source with toluene in the presence of a catalyst disposed within a reactor to form a product stream including ethylbenzene, wherein the catalyst includes a nanocrystalline zeolite; and recovering the product stream from the reactor.

One or more embodiments include the process of the preceding paragraph, wherein the nanocrystalline zeolite includes a particle size of less than about 1000 nm.

One or more embodiments include the process of any preceding paragraph, wherein the nanocrystalline zeolite includes a particle size of less than about 300 nm.

One or more embodiments include the process of any preceding paragraph, wherein the nanocrystalline zeolite is formed of an X-type zeolite.

One or more embodiments include the process of any preceding paragraph, wherein the catalyst further includes a metal selected from Ru, Rh, Ni, Co, Pd, Pt, Mn, Ti, Zr, V, Nb, K, Cs, Ga, Ph, B and Na and combinations thereof.

One or more embodiments include the process of any preceding paragraph, wherein the catalyst further includes a support material.

One or more embodiments include the process of any preceding paragraph, wherein the support material is selected from silica, alumina, aluminosilica, titania, zirconia and combinations thereof.

One or more embodiments include the process of any preceding paragraph, wherein the product stream further includes styrene.

One or more embodiments include the process of any preceding paragraph further including converting a C₁ source to form an intermediate product selected from formaldehyde, hydrogen, water, methanol and combinations thereof.

One or more embodiments include the process of any preceding paragraph, wherein the C₁ source is selected from methanol, formaldehyde, formalin, trioxane, methylformcel, paraformaldehyde and methyal and combinations thereof.

One or more embodiments include the process of any preceding paragraph, wherein the C₁ source includes a mixture of methanol and formaldehyde.

One or more embodiments include the process of any preceding paragraph, wherein toluene conversion is greater than 0.1 mol %.

One or more embodiments include the process of any preceding paragraph, wherein toluene conversion is greater than 15 mol %.

One or more embodiments include the process of any preceding paragraph, wherein selectivity to styrene is greater than 2 mol % and selectivity to ethylbenzene is greater than 10 mol %.

### BRIEF DESCRIPTION OF DRAWINGS

FIG. 1 illustrates a now chart for a styrene production process.

FIG. 2 illustrates a flow chart for alternative styrene production process.

### DETAILED DESCRIPTION

### Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions when the information in this patent is combined with available information and technology.

Various terms as used herein are shown below. To the extent a term used in a claim is not defined below, it should he given the broadest definition skilled persons in the pertinent art have given that term as reflected in printed publications and issued patents at the time of filing. Further, unless otherwise specified, all compounds described herein may he substituted or unsubstituted and the listing of compounds includes derivatives thereof.

Further, various ranges and/or numerical limitations may be expressly stated below. It should he recognized that unless stated otherwise, it is intended that endpoints are to be interchangeable. Further, any ranges include iterative ranges of like magnitude falling within the expressly stated ranges or limitations.

Styrene production processes generally include reacting toluene with methanol or methane/oxygen as co-feed. In practice, the methanol (CH₃OH) often dehydrogenates into side-products, resulting in lower than desired toluene conversion and/or lower than desired selectivity. As used herein, the term “selectivity” refers to the percentage of input/reactant converted to a desired output/product. Such low conversion/selectivity rates generally lead to processes which are not economical.

However, the processes described herein (and particularly the catalysts described herein in combination with the described processes) are capable of minimizing side product formation, thereby resulting in increased conversion and/or selectivity.

In one or more embodiments, the styrene production processes include reacting toluene with a carbon source, which can be referred to as a C₁ source (e.g., a carbon source capable of cross-coupling with toluene to form styrene, ethylbenzene or combinations thereof), in the presence of a catalyst to produce a product stream including styrene and ethylbenzene. For example, the C₁ source may include methanol, formaldehyde or a mixture thereof. Alternatively, the C₁ source includes toluene reacted with a C₁ source selected from one or more of the following: formalin (37 wt. % to 50 wt. % H₂CO in a solution of water and MeOH), trioxane (1,3,5-trioxane), methylformcel (55 wt. % H₂CO in methanol), paraformaldehyde and methyal (dimethoxymethane). In another embodiment, the C₁ source is selected from methanol, formaldehyde, formalin, trioxane, methylformcel, paraformaldehyde, methyal and combinations thereof.

Formaldehyde can be produced by the oxidation or dehydrogenation of methanol, for example. In one embodiment, formaldehyde is produced by the dehydrogenation of methanol to produce formaldehyde and hydrogen gas. This reaction step generally produces a dry formaldehyde stream, thereby eliminating separation of formed water prior to the reaction of the formaldehyde with toluene. The dehydrogenation process is described in the equation below:

CH₃OH→CH₂O+H₂.

Formaldehyde can also be produced by the oxidation of methanol to produce formaldehyde and water. The oxidation of methanol is described in the equation below:

2 CH₃OH+O₂→2 CH₂O+2 H₂O.

When utilizing a separate process to obtain formaldehyde, a separation unit may then be used to separate the formaldehyde from the hydrogen gas or water from the formaldehyde and unreacted methanol prior to reacting the formaldehyde with toluene for the production of styrene. Such separation inhibits hydrogenation of the formaldehyde hack to methanol. Purified formaldehyde can then be sent to a styrene reactor and the unreacted methanol recycled, for example.

Although the equations illustrated above show a 1:1 molar ratio of toluene and the C, source, such molar ratio is not limited within the embodiments herein and can vary depending on operating conditions and efficiency of the reaction system. For example, if excess toluene or C₁ source is fed to the reaction zone, the unreacted portion can be subsequently separated and recycled back into the process. In one embodiment the molar ratio of toluene:C₁ source can range from 100:1 to 1:100. In alternate embodiments, the molar ratio of toluene:C₁ source can range from 50:1 to 1:50, or from 20:1 to 1:20, or from 10:1 to 1:10, or from 5:1 to 1:5 or from 2:1 to 1:2, for example.

The styrene production process generally includes catalyst disposed within one or more reactors. The reactors may include fixed bed reactors, fluid bed reactors, entrained bed reactors or combinations thereof, for example. Reactors capable of operation at the elevated temperature and pressure as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to he limiting on the scope of the present invention.

In another aspect, the one or more reactors may include one or more catalyst beds. When utilizing multiple beds, an inert material layer may separate each bed. The inert material may include any type of inert substance, such as quartz, for example. In one or more embodiments, the reactor includes from 1 to 10 catalyst beds or from 2 to 5 catalyst beds, for example. In addition, the C₁ source and toluene may be injected into a catalyst bed, an inert material layer or combinations thereof, for example. Alternatively, at least a portion of the C₁ source may be injected into a catalyst bed(s) and at least a portion of the toluene feed is injected into an inert material layer(s). In yet another embodiment, the entire C₁ source may be injected into a catalyst bed(s) and all of the toluene feed is injected into an inert material layer(s). Alternatively, at least a portion of the toluene feed may be injected into a catalyst beds) and at least a portion the C₁ source may be injected into an inert material layer(s). In yet another embodiment, all of the toluene feed may be injected into a catalyst bed(s) and the entire C₁ source may be injected into an inert material layer(s).

The operating conditions of the reactors will he system specific and can vary depending on the feedstream composition and the composition of the product streams. In one or more embodiments, the reactors) may operate at elevated temperatures and pressures, for example.

In one or more embodiments, the elevated temperature can range from 250° C. to 750° C., or from about 300° C. to about 500° C. or from about 325° C. to about 450° C., for example. The elevated pressure can range from from 1 atm to 70 atm, or from 1 atm to about 35 atm or from about 1 atm to about 5 atm, for example.

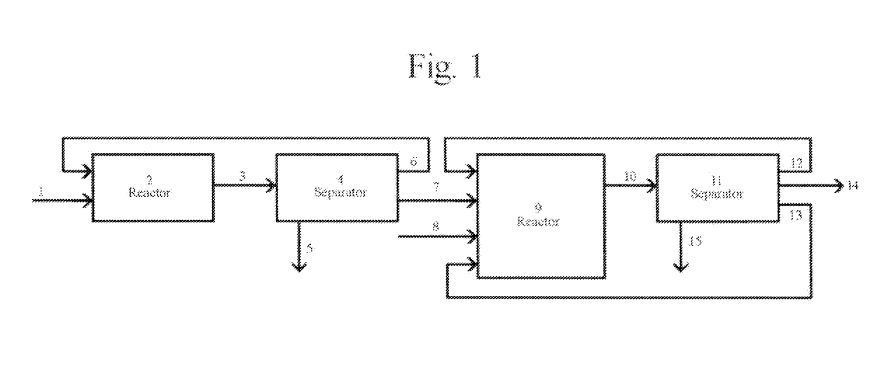

FIG. 1 illustrates a simplified flow chart of one embodiment of the styrene production process described above wherein the C₁ source is formaldehyde. In this embodiment, a first reactor (2) is either a dehydrogenation reactor or an oxidation reactor. First reactor (2) is designed to convert a first methanol feed (1) into formaldehyde. The product stream (3) of the first reactor (2) may then be sent to an optional gas separation unit (4) where the formaldehyde is separated from any unreacted methanol (6) and unwanted byproducts (5). Any unreacted methanol (6) can then be recycled back into the first reactor (2). The byproducts (5) are separated from the clean formaldehyde (7).

In one embodiment, the first reactor (2) is a dehydrogenation reactor that produces formaldehyde and hydrogen and the gas separation unit (4) is a membrane capable of removing hydrogen from the product stream (3).

In an alternate embodiment, the first reactor (2) is an oxidative reactor that produces product stream (3) comprising formaldehyde and water. The product stream (3) comprising formaldehyde and water can then he sent to the second reactor (9) without a gas separation unit (4).

The clean formaldehyde (7) is then reacted with a feed stream of toluene (8) in the second reactor (9) in the presence of a catalyst (not shown) disposed in the second reactor (9). The toluene and formaldehyde react to produce styrene. The product (10) of the second reactor (9) may then be sent to an optional separation unit (11) where any unwanted byproducts (15), such as water, can be separated from the styrene, unreacted formaldehyde (12) and unreacted toluene (13). Any unreacted formaldehyde (12) and unreacted toluene (13) can be recycled hack into the second reactor (9). A styrene product stream (14) can be removed from the separation unit (11) and subjected to further treatment or processing if desired.

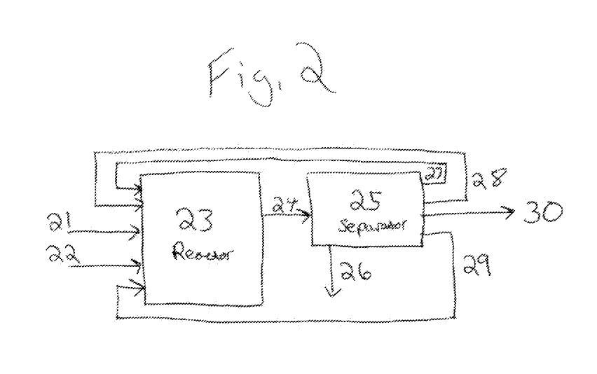

FIG. 2 illustrates a simplified flow chart of another embodiment of the styrene process discussed above wherein the C1 source is methanol. A methanol containing ked stream (21) is fed along with a feed stream of toluene (22) to a reactor (23) having a catalyst (not shown) disposed therein. The methanol reacts with the catalyst to produce a product (24) including styrene. The product (24) of the reactor (23) may then be sent to an optional separation unit (25) where any unwanted byproducts (26) can be separated from the styrene. unreacted methanol (27), unreacted formaldehyde (28) and unreacted toluene (29). Any unreacted methanol (27), unreacted formaldehyde (28) and unreacted toluene (29) can be recycled back into the reactor (23). A styrene product stream (30) can be removed from the separation unit (25) and subjected to further treatment or processing if desired.

The catalyst utilized for the processes described herein generally includes a zeolitic material. As used herein, the term “zeolitic material” refers to a molecular sieve containing an alumino silicate lattice. Zeolitic materials are well known in the art and possess well-arranged pore systems with uniform pore sizes. However, these materials tend to possess either only micropores or only mesopores, in most cases only micropores. Micropores are defined as pores having a diameter of less than about 2 nm. Mesopores are defined as pores having a diameter ranging from about 2 nm to about 50 nm. Micropores generally limit external molecules access to the catalytic active sites inside of the micropores or slow down diffusion to the catalytic active sites.

However, embodiments of the invention utilize a nanocrystalline zeolite. As used herein, the term “nanocrystalline zeolite” refers to zeolitic materials having a particle size smaller than 1000 nm. For example, the particle size may be less than 1000 nm, or less than 300 nm, or less 100 nm, or less than 50 nm or less than 25 nm, for example. In one or more embodiments, the particle size is from 25 nm to 300 nm, or from 50 nm to 100 nm or from 50 nm to 75 nm, for example. As used herein, the “particle size” refers to either the size of each discrete crystal (i.e., crystal) of the zeolitic material or the size of an agglomeration of particles (i.e., crystallite) within the zeolitic material.

The zeolitic materials may include silicate-based zeolites, such as faujasites and mordenites, for example. Silicate-based zeolites may he formed of alternating SiO₂ and MOₓ tetrahedra, where M is an element selected from the Groups 1 through 16 of the Periodic Table. Such formed zeolites may have 4, 6, 8, 10, or 12-membered oxygen ring channels, for example. Other suitable zeolite materials include X-type and Y-type zeolites. As used herein the term “X-type” refers to zeolitic materials having a silicon:aluminum molar ratio of from 1:1 to 1.7:1 and “Y-type” refers to zeolitic materials having a silicon:aluminum molar ratio of greater than 1.7:1.

The catalyst generally includes from about 0.1 wt. % to about 99 wt. %, or from 3 wt. % to about 90 wt. % or from about 4 wt. % to about 80 wt. % nanocrystalline zeolite, for example.

In one or more embodiments, the nanocrystalline zeolite may have an increase ratio of surface area to volume of compared to zeolitic materials that are not nanocrystalline, for example.

The nanocrystalline zeolite may be supported by methods known to one skilled in the art. For example, such methods may include impregnating a solid, porous alumnio silicate particle or structure with a concentrated aqueous solution of an inorganic micropore-forming directing agent through incipient wetness impregnation. Alternatively, the nanocrystalline zeolite can be admixed with a support material, for example. It is further contemplated that the nanocrystalline zeolite may he supported in-situ with the support material or extruded, for example. Alternatively, the nanocrystalline zeolite may be supported by spray-coating the nonacrystalline material onto a support material. It is further contemplated that such support processes may include layering the nanocrystalline zeolite onto the support material, such as the support materials described below or optionally polymer spheres, such as polystyrene spheres, for example. It is further contemplated that such support processes may include the utilization of zeolitic membranes, for example.

In one specific embodiment, the nanocrystalline zeolite is supported by incipient wetness impregnation. Such process generally includes dispersing the nanocrystalline zeolite in a diluent, such as methanol, to yield individual crystals. A support material may then be added to the solution and mixed until dry.

In yet another embodiment. the nanocrystalline zeolite is supported by forming a mini extrusion batch utilizing a support material in combination with the nanocrystalline zeolite to form extrudates.

Optional support materials may include silica, alumina, aluminosilica, titania, zirconia and combinations thereof, for example. In one or more embodiments, the catalyst includes from about 5 wt. % to about 20 wt. %, or from about 5 wt. % to about 15 wt. % or from about 7 wt. % to about 12 wt. % support material, for example.

The catalysts described herein increase the effective diffusivity of the reactants, thereby increasing reactant conversion to desired products. Furthermore, the catalysts result in processes exhibiting improved product selectivity over processes utilizing conventional zeolitic materials.

In addition, activity of such processes is increased due to an increase of accessibility of inner active sites, which thereby increases the effective number of active sites per weight of catalyst over larger non nanocrystalline zeolites. As used herein, the term “activity” refers to the weight of product produced per weight of the catalyst used in a process at a standard set of conditions per unit time.

Optionally, a catalytically active metal may he incorporated into the nanocrystalline zeolite by, for example, ion-exchange or impregnation of the zeolitic material, or by incorporating the active metal in the synthesis materials from which the zeolitic material is prepared. As described herein, the term “incorporated into the zeolitic material” refers to incorporation into the framework of the zeolitic material, incorporation into channels of the zeolitic material (i.e., occluded) or combinations thereof.

The catalytically active metal can be in a metallic form, combined with oxygen (e.g., metal oxide) or include derivatives of the compounds described below, for example. Suitable catalytically active metals depend upon the particular process in which the catalyst is intended to be used and generally include, but are not limited to, alkali metals (e.g., Li, Na, K, Ru, Cs, Fr), rare earth “lanthanide” metals (e.g., La, Ce, Pr), Group IVB metals (e.g., Ti, Zr, Hf), Group VB metals (e.g., V, Nb, Ta), Group VIB metals (e.g., Cr, Mo, W), Group IB metals (e.g., Cu, Ag, Au), Group VIIIB metals (e.g., Pd, Pt, Ir, Co, Ni, Rh, Os, Fe), Group IIIA metals (e.g., Ga) and combinations thereof, for example. Alternatively (or in combination with the previously discussed metals), the catalytically active metal may include a Group IIIA compound (e.g., B), Group VA compound (e.g., P) or combinations thereof, for example. In one or more embodiments, the catalytically active metal is selected from Cs, Na, B, Ga and combinations thereof.

In one or more embodiments, the nanocrystalline zeolite may include less than about 10 wt. % sodium, for example. In one or more embodiments, the nanocrystalline zeolite may include less than about 5 wt. % aluminum, for example. In one or more embodiments, the nanocrystalline zeolite may include at least about 30 wt. % cesium, for example. In one or more embodiments, the nanocrystalline zeolite may include at least about 10 wt. % silicon, for example. In one or more embodiments, the nanocrystalline zeolite may include at least about 0.1 wt. % boron, for example it is to be recognized that the balance of the nanocrystalline zeolite will be formed of oxygen.

Furthermore, increased side chain alkylation selectivity towards desired products may be achieved by treating the catalyst with chemical compounds to inhibit basic sites. Such improvement may be accomplished by the addition of a second metal. The second metal can be one of those mentioned above. For example, in one or more embodiments, the second metal may include boron.

It is further contemplated that the zeolitic material, the catalytically active metal, the support material or combinations thereof may optionally be contacted with a carrier prior to contact of the zeolitic material with the catalytically active metal. Such carrier may be adapted to aid in the incorporation of the catalytically active metal into the zeolitic material, for example. In one or more embodiments, the carrier includes aluminum, for example. In one or more embodiments, the carrier is a nano-sized carrier (with the nano-sized carrier defined as for nanocrystalline zeolites, as described above).

In one embodiment, the nanocrystalline zeolite is formed by utilizing a carrier to transport the nanocrystalline zeolite into pores of the support material. The formed zeolite may then be dried, for example. It is further contemplated that the carrier may be mixed with a solvent prior to contact with the nanocrystalline zeolite.

The processes described herein may exhibit a toluene conversion of at least 0.01 mol. %, or from 0.05 mol. % to 40 mol. %, or from 2 mol. % to 25 mol. % or from 5 mol. % to 25 mol. %, for example.

The process may exhibit a selectivity to styrene of at least 1 mol. %, or from 1 mol. % to 99 mol. % or at least 30 mol. % or from 65 mol % to 99 mol %, for example.

The process may exhibit a selectivity to ethylbenzene of at least 5 mol. %, or from 5 mol. % to 99 mol. %, or at least 10 mol. % or from 8 mol % to 99 mol %, for example.

While the foregoing is directed to embodiments of the present invention, other and further embodiments of the invention may he devised without departing from the basic scope thereof and the scope thereof is determined by the claims that follow.

What is claimed is:

1. A styrene production process comprising:
providing a C₁ source;
contacting the C₁ source with toluene in the presence of a catalyst disposed within a reactor to form a product stream comprising ethylbenzene, wherein the catalyst comprises a nanocrystalline zeolite; and
recovering the product stream from the reactor.

2. The process of claim 1, wherein the nanocrystalline zeolite comprises a particle size of less than about 1000 nm.

3. The process of claim 1, wherein the nanocrystalline zeolite comprises a particle size of less than about 300 nm.

4. The process of claim 1, wherein the nanocrystalline zeolite is formed of an X-type zeolite.

5. The process of claim 1, wherein the catalyst further comprises a metal selected from Ru, Rh, Ni, Co, Pd, Pt, Mn, Ti, Zr, V, Nb, K, Cs, Ga, Ph, B and Na and combinations thereof.

6. The process of claim 1, wherein the catalyst further comprises a support material.

7. The process of claim 6, wherein the support material is selected from silica, alumina, aluminosilica, titania, zirconia and combinations thereof.

8. The process of claim 1, wherein the product stream further comprises styrene.

9. The process of claim 1 further comprising converting a C₁ source to form an intermediate product selected from formaldehyde, hydrogen, water, methanol and combinations thereof.

10. The process of claim 1, wherein the C₁ source is selected from methanol, formaldehyde, formalin, trioxane, methylformcel, paraformaldehyde and methyal and combinations thereof.

11. The process of claim 10, wherein the C₁ source comprises a mixture of methanol and formaldehyde.

12. The process of claim 1, wherein toluene conversion is greater than 0.1 mol %.

13. The process of claim 1, wherein toluene conversion is greater than 15 mol %.

14. The process of claim 1, wherein selectivity to styrene is greater than 2 mol % and selectivity to ethylbenzene is greater than 10 mol %.
